(19) United States
(12) Patent Application Publication
(10) Pub. No.: US 2022/0372560 A1
(43) Pub. Date: Nov. 24, 2022

(54) METHODS FOR DETECTING OLIGONUCLEOTIDES

(71) Applicant: Takeda Pharmaceutical Company Limited, Osaka (JP)

(72) Inventors: Yinghua Shen, Lexington, MA (US); Dennis Keefe, Bedford, MA (US); Deborah Palliser, Cambridge, MA (US)

(21) Appl. No.: 17/773,535

(22) PCT Filed: Oct. 29, 2020
(86) PCT No.: PCT/US20/58044

§ 371 Date: Apr. 29, 2022

Related U.S. Application Data
(60) Provisional application No. 62/928,301, filed on Oct. 30, 2019.

(51) Int. Cl. C12Q 1/6816

(52) U.S. Cl. CPC C12Q 1/6816

(57) ABSTRACT

The present disclosure describes methods for detecting oligonucleotide presence and/or quantity in a sample.

### CROSS-REFERENCE TO RELATED APPLICATIONS

This application claims the benefit of U.S. Provisional Application 62/928,301, filed Oct. 30, 2019, the contents of which are hereby incorporated by reference in their entirety.

### BACKGROUND

A number of diseases may benefit from treatment comprising administration of oligonucleotides such as antisense oligonucleotides. For example, in a disorder such as Duchenne Muscular Dystrophy (DMD) in which the dystrophin gene has one or more mutations that result in dysfunctional or absent dystrophin protein, use of oligonucleotides (e.g., antisense oligonucleotides) to “skip” over certain portions of a genetic sequence during dystrophin protein production result in a truncated, but partially functional, dystrophin protein. However, development of efficacious oligonucleotides, as well as accurate testing and modeling for efficacious dose projection suffers from an unmet need for high-throughput, sensitive, accurate, and reproducible methods to detect oligonucleotides which have been administered to a subject in need thereof. Such methods are, for example, needed to accurately and reproducibly detect, as well as quantitate, oligonucleotides in samples from subjects to whom they have been administered.

### SUMMARY

The present disclosure is based in part on the development of an improved assay that allows sensitive detection of oligonucleotides in samples from subjects to whom they have been administered.

In some aspects, the present disclosure provides a method comprising steps of: (a) incubating a sample comprising oligonucleotides with an oligonucleotide probe, wherein the oligonucleotide probe hybridizes with the oligonucleotides in the sample; (b) incubating the product of step (a) with a substrate coated with a capture agent, wherein the capture agent binds the oligonucleotide probe thereby causing the excess oligonucleotide probe and hybridized oligonucleotides to become associated with the substrate, and optionally washing the plate after the hybridized probes become associated with the substrate; (c) incubating the substrate after step (b) with two or more different single-strand-specific nucleases that have a different specificity for the substrate, wherein the substrate is optionally washed with one or more wash solutions before, during or after step (c), and the oligonucleotide probe and hybridized oligonucleotides remain associated with the substrate during the washing step or steps; (d) incubating the substrate after step (c) with a detection agent, wherein the detection agent interacts with the oligonucleotide probe to produce a detectable signal; and (e) detecting the detectable signal.

In some aspects, the present disclosure provides a method comprising steps of: (a) incubating a sample comprising phosphorodiamidate morpholino oligonucleotides (PMOs) with an oligonucleotide probe, wherein the oligonucleotide probe comprises one or more locked nucleic acid (LNA) residues and hybridizes with PMOs in the sample; (b) incubating the product of step (a) with a substrate coated with a capture agent, wherein the capture agent binds the oligonucleotide probe thereby causing the oligonucleotide probe and hybridized PMOs to become associated with the substrate; (c) incubating the substrate after step (b) with one or more single-strand-specific nucleases, wherein the substrate is optionally washed before, during or after step (c) and the oligonucleotide probe and hybridized PMOs remain associated with the substrate during the washing step or steps; (d) incubating the substrate after step (c) with a detection agent, wherein the detection agent interacts with the oligonucleotide probe to produce a detectable signal; and (e) detecting the detectable signal.

In some embodiments, step (c) comprises incubating the substrate with micrococcal nuclease and mung bean nuclease.

In some embodiments, step (c) comprises incubating the substrate with the micrococcal nuclease and then incubating the substrate with the mung bean nuclease.

In some embodiments, the substrate is washed after it has been incubated with the micrococcal nuclease and before it is incubated with the mung bean nuclease.

In some embodiments, the sample comprises phosphorodiamidate morpholino oligonucleotides (PMOs).

In some embodiments, the oligonucleotide probe comprises one or more locked nucleic acids (LNA) residues.

In some embodiments, the oligonucleotide probe comprises one or more deoxyribonucleic acid (DNA) residues.

In some embodiments, the oligonucleotide probe comprises a central segment which is comprised of DNA residues flanked by 5′ and 3′ terminal segments which each comprise LNA residues.

In some embodiments, the central segment is comprised of between 5 and 20 DNA residues.

In some embodiments, the 5′ and 3′ terminal segments are independently comprised of between 2 and 8 LNA residues.

In some embodiments, step (c) comprises incubating the substrate with only one single-strand-specific nuclease.

In some embodiments, step (c) comprises incubating the substrate with two or more different single-strand-specific nucleases.

In some embodiments, the substrate is incubated with the two or more different single-strand-specific nucleases sequentially and the substrate is washed after each incubation.

In some embodiments, the substrate is incubated with the two or more single-strand-specific nucleases simultaneously.

In some embodiments, the one or more single-strand-specific nucleases comprises micrococcal nuclease.

In some embodiments, the one or more single-strand-specific nucleases comprises mung bean nuclease.

In some embodiments, the one or more single-strand-specific nucleases comprises micrococcal nuclease and mung bean nuclease.

In some embodiments, the only one single-strand-specific nuclease is mung bean nuclease.

In some embodiments, the two or more different nucleases comprise micrococcal nuclease and mung bean nuclease.

In some embodiments, the sample is obtained from a tissue and the method further comprises a step of quantifying a level of PMO in the tissue based on a level of the detectable signal detected in step (e).

In some embodiments, the tissue is selected from blood, kidney, liver, gastrointestinal, lung, muscle, spleen, brain, spinal cord, or a combination thereof.

In some embodiments, the blood tissue is or comprises plasma.

In some embodiments, the muscle tissue is diaphragm, gastrocnemius, tibialis anterior (TA), biceps, heart, and/or quadriceps.

In some embodiments, the sample comprises a PMO which comprises a sequence of 20 to 30 contiguous nucleotides and has a nucleotide sequence selected from at least one exon of a mammalian dystrophin gene.

In some embodiments, the at least one exon is selected from 23, 44, 45, 46, 51, or 53.

In some embodiments, the mammalian dystrophin gene is a human dystrophin gene.

In some embodiments, the PMO has previously been delivered to a patient, and wherein the PMO was conjugated to a peptide (P-PMO) for delivery.

In some aspects, the present disclosure provides a method of assessing the tissue distribution of a P-PMO, the method comprising performing the method of any one of the preceding claims on one or more samples that have been obtained from one or more tissues of a subject to whom the P-PMO has been administered.

In some embodiments, the method is performed on two or more samples that have been obtained from two or more tissues of the subject.

In some embodiments, the method is repeated for a different P-PMO.

In some aspects, the present disclosure provides a method of assessing the ability of a P-PMO to modulate expression of a target protein, the method comprising performing the method of any one of the preceding claims on a sample that has been obtained from a tissue of a subject to whom a P-PMO has been administered and comparing the level of a PMO derived from the P-PMO in the tissue with a level of the target protein in the tissue.

In some embodiments, the method is performed on two or more samples that have been obtained from two or more tissues of the subject.

In some embodiments, the method is repeated for a different P-PMO.

In some embodiments, the target protein is dystrophin.

### BRIEF DESCRIPTION OF THE DRAWING

FIG. 1 is a flow chart of an exemplary ELISA-based detection assay according to an embodiment described herein.

FIGS. 2a-2e show an exemplary oligonucleotide design scheme and assays comparing certain exemplary annealing and digestion conditions for oligonucleotide probes as described herein. FIG. 2a shows an exemplary oligonucleotide probe design scheme for exemplary oligonucleotides used in a detection assay as described herein. Phosphorothioate (“PTO”)-modified bases are highlighted in bold, and the locked nucleic acid (“LNA”)-modified bases are shown in bold and underlined. FIGS. 2b and 2c show comparisons of levels of oligonucleotide detected using PTO/DNA and LNA/DNA oligonucleotide probes. FIG. 2d shows levels of oligonucleotide detected using LNA/DNA oligonucleotide probes and an assay performed at annealing temperatures of 37° C. or 50° C. FIG. 2e shows a comparison of LNA/DNA oligonucleotide probes that were incubated without mung bean nuclease or with mung bean nuclease at pH 5 or pH 7. Concentration of nucleotide that was “spiked in” to the assay is shown on the x-axis (in pM) and signal (in RFU) is shown on the y-axis of FIGS. 2b-2e.

FIGS. 3a and 3b show validation of an exemplary detection assay as described herein using an LNA/DNA oligonucleotide probe. FIG. 3a shows qualification of an exemplary detection assay in two different mouse serum samples. The bar graph shows measured concentration of oligonucleotide on the y-axis and concentration of oligonucleotide in a prepared standard solution on the x-axis in each serum sample. FIG. 3b shows a representative standard curve obtained by measuring oligonucleotide concentration in mouse serum. Open squares and circles show back-calculated PMO concentrations. Appearance of straight line indicates 100% recovery when comparing a prepared standard and detectable levels of oligonucleotide measured in mouse serum. All samples were run in duplicate.

FIGS. 4a-4j show detection of oligonucleotide in mouse serum and tissues from mice injected with oligonucleotide. Mice were injected IV with a single bolus of a composition comprising an oligonucleotide (e.g., P-PMO A) at 4.4 mg/kg and serum was analyzed at various time points as seen on the x-axis indicated for concentration (in pM) of P-PMO A (FIG. 4a) or P-PMO B (FIG. 4b). The left panels in FIGS. 4a and 4b show the entire time course; the right panels in FIGS. 4a and 4b show a split x-axis, where the first 8 hours are expanded relative to the right panel. FIGS. 4c-4j show levels of oligonucleotide detected (in pmoles/g tissue) in heart (FIG. 4c), diaphragm (FIG. 4d), gastrocnemius (FIG. 4e), Tibialis Anterior (“TA”) (FIG. 4f), lung (FIG. 4g), kidney (FIG. 4h), liver (FIG. 4i), and spleen (FIG. 4j). In FIGS. 4c-4j, solid black circles=P-PMO A and open squares=P-PMO B.

FIGS. 5a and 5b show measured concentrations (in pM) of oligonucleotide not conjugated to a peptide (PMO) and oligonucleotide conjugated to a peptide (P-PMO A and P-PMO B). FIG. 5a shows absolute concentrations of oligonucleotide detected in serum and various muscle-containing tissues from mice administered a given oligonucleotide. FIG. 5b shows concentrations of oligonucleotides normalized to total moles of oligonucleotide (e.g., PMO or P-PMO) dosed.

FIGS. 6a-6e show oligonucleotide concentration in mice, over time, after administration of 6 mg/kg, 12 mg/kg, 20 mg/kg, or 40 mg/kg oligonucleotide (as P-PMO) as measured in serum (FIG. 6a), heart (FIG. 6b), diaphragm (FIG. 6c), TA (FIG. 6d), and quadriceps (FIG. 6e).

### DEFINITIONS

In this application, unless otherwise clear from context, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; and (iv) the terms “about” and “approximately” may be understood to permit standard variation as would be understood by those of ordinary skill in the art; and (v) where ranges are provided, endpoints are included.

Administration: As used herein, the term “administration” typically refers to the administration of a composition to a subject or system to achieve delivery of an agent that is, or is included in, the composition. Those of ordinary skill in the art will be aware of a variety of routes that may, in appropriate circumstances, be utilized for administration to a subject, for example a human. For example, in some embodiments, administration may be ocular, oral, parenteral, topical, etc. In some particular embodiments, administration may be bronchial (e.g., by bronchial instillation), buccal, dermal (which may be or comprise, for example, one or more of topical to the dermis, intradermal, interdermal, transdermal, etc.), enteral, intra-arterial, intradermal, intragastric, intramedullary, intramuscular, intranasal, intraperitoneal, intrathecal, intravenous, intraventricular, within a specific organ (e.g., intrahepatic), mucosal, nasal, oral, rectal, subcutaneous, sublingual, topical, tracheal (e.g., by intratracheal instillation), vaginal, vitreal, etc. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time. In some embodiments, administration may depend on one or more results of a diagnostic or monitoring assay to determine, for example, concentration of a particular administered agent in a sample (e.g., tissue, serum, etc.) from a subject to whom the agent was administered.

Nuclease: As used herein, the term “nuclease” refers to a polypeptide capable of cleaving bonds. In some embodiments, a bond is phosphodiester bonds between the nucleotide subunits of nucleic acids. In some embodiments, a bond is between a peptide and an oligonucleotide. In some embodiments, a nuclease is a single-strand-specific nuclease. In some embodiments, a nuclease is a mung bean nuclease. In some embodiments, a nuclease is a micrococcal nuclease.

Nucleic acid: As used herein, in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to an individual nucleic acid residue (e.g., a nucleotide and/or nucleoside); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, “nucleic acid” refers to a nucleic acid molecule. For example, in some such embodiments, nucleic acid may refer to a polymer of deoxyribonucleotides or ribonucleotides in either single- or double-stranded form, comprising nucleotides or analogs thereof. Such a nucleic acid may also be referred to and/or used interchangeably with the term “polynucleotide.” In some embodiments, a nucleic acid is partly or wholly single stranded; in some embodiments, a nucleic acid is partly or wholly double stranded. In some embodiments, a nucleic acid is, comprises, or consists of one or more natural nucleic acid residues. In some embodiments, a nucleic acid is, comprises, or consists of one or more synthetic nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. For example, in some embodiments, a nucleic acid is, comprises, or consists of one or more “locked nucleic acids”, which are known in the art. Alternatively or additionally, in some embodiments, a nucleic acid has one or more phosphorothioate and/or 5′-N-phosphoramidite linkages rather than phosphodiester bonds. In some embodiments, a nucleic acid is or comprises a phosphorodiamidate morpholino oligonucleotide (PMO). In some embodiments, an oligonucleotide is a phosphorothioate-linked 2′-O-methyl RNA (2′OMeP). In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments a nucleic acid has a nucleotide sequence comprising at least one element that encodes, or is the complement of a sequence that encodes, a polypeptide.

Oligonucleotides: As used herein, the term “oligonucleotide” refers to a polymer of nucleic acids that may be designed with the intention of and/or may be administered to a subject in need thereof. In some embodiments, an oligonucleotide is, comprises, or functions as an antisense oligonucleotide (ASO). In some embodiments, an oligonucleotide is 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or more nucleic acid bases long. In some embodiments, an oligonucleotide is or comprises more than one type of nucleic acid as described herein. In some embodiments, an oligonucleotide is an ASO, which, when administered to a subject in need thereof, may facilitate production of a target protein or functional portion thereof that is otherwise not made in the absence of the ASO. In some embodiments, an oligonucleotide is a phosphorodiamidate morpholino oligonucleotide (PMO). In some embodiments, an oligonucleotide is conjugated to a peptide (e.g., a P-PMO). In some embodiments, an oligonucleotide is a phosphorothioate-linked 2′-O-methyl RNA (2′ OMeP).

Oligonucleotide probe: As used herein, the term “oligonucleotide probe” refers to a polymer of nucleic acids that may be used in an assay (e.g., a detection assay). In the present disclosure, an oligonucleotide probe may also be referred to and used interchangeably with the term “probe”. In some embodiments an oligonucleotide probe used in an assay designed to detect presence or absence of one or more oligonucleotides or candidate oligonucleotides. For example, in some embodiments, an oligonucleotide probe contacts a sample comprising an oligonucleotide. In some such embodiments, methods as described herein are used to determine presence and, if appropriate, quantity of the oligonucleotide that the oligonucleotide probe is designed to detect. In some embodiments, an oligonucleotide probe is or comprises deoxyribonucleotides or ribonucleotides in either single- or double-stranded form, comprising nucleotides or analogs thereof. In some embodiments, an oligonucleotide probe is or comprises more than one type of nucleic acid, as described herein. For example, in some embodiments, an oligonucleotide probe is or comprises phosphorothioate nucleic acids (“PTO”) and DNA (PTO/DNA). In some embodiments, an oligonucleotide probe comprises locked nucleic acids and DNA (LNA/DNA). In some such embodiments, PTOs or LNAs are localized to the 5′ and 3′ ends of a provided oligonucleotide probe. In some embodiments, the number of PTO or LNA residues on the 5′ and 3′ ends of a provided oligonucleotide probe is not the same. For example, in some embodiments, a 5′ end of an oligonucleotide probe may have three LNAs and its 3′ end may have five or six LNAs. In some embodiments, a particular combination of nucleotides in an oligonucleotide probe (e.g., LNA/DNA) will provide one or more advantages in an assay (e.g., a detection assay) as compared to other combinations of nucleotides (e.g., PTO/DNA). In some embodiments, an oligonucleotide is at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 nucleic acid bases long.

Single Strand Specific Nuclease: As used herein, the term “single-strand specific nuclease” refers to a nuclease that preferentially cleaves a bond in a single-stranded nucleic acid polymer. In some embodiments, a nucleic acid polymer is an oligonucleotide probe. In some such embodiments, the oligonucleotide probe is present in a mixture, in an excess amount, and a single-strand specific nuclease cleaves the probe for washing and/or removal. In some such embodiments, more than one nuclease is used, either sequentially, or simultaneously. In some embodiments, a single-strand specific nuclease is a mung bean nuclease. In some embodiments, a single-strand specific nuclease is micrococcal nuclease.

Subject: As used herein, the term “subject” refers an organism, typically a mammal (e.g., a human, in some embodiments including prenatal human forms). In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

### DETAILED DESCRIPTION

The present disclosure describes methods of detecting an oligonucleotide in a sample of a subject that was administered an oligonucleotide. In particular, the present disclosure describes methods of quantifying and/or localizing one or more oligonucleotides in one or more samples from subjects to whom the one or more oligonucleotides was administered. The methods described herein provide unexpectedly sensitive, accurate, and reproducible methods of detection as compared to previously available assays.

### Methods of Detection

The present disclosure provides, among other things, methods of detecting oligonucleotides in a given sample. These methods of detecting are much more sensitive, accurate, and reproducible than any previously available assays for detecting oligonucleotides as described herein. In some embodiments, an assay used for detection of an oligonucleotide comprises steps of incubating with an oligonucleotide probe, incubating a mixture comprising an oligonucleotide and an oligonucleotide probe with a surface comprising a capture agent, performing one or more digestion steps on the mixture incubated with the surface, followed by performing one or more steps comprising contacting the sample with a substrate and a detection agent to detect and/or quantify an oligonucleotide in a sample.

In some embodiments, a sample comprises an oligonucleotide. In some embodiments, the oligonucleotide is a PMO. In some embodiments, a sample comprises serum or tissue to which an oligonucleotide was added, in vitro. In some embodiments, a sample comprises serum or tissue from a subject to whom an oligonucleotide was administered. In some embodiments, a sample comprises serum or tissue from a subject to whom an oligonucleotide was not administered.

In some embodiments, presence and/or quantity of an oligonucleotide is detected using an oligonucleotide probe.

In some embodiments, incubating a sample comprises oligonucleotides with an oligonucleotide probe results in hybridization of an oligonucleotide probe with a portion of an oligonucleotide.

In some embodiments, a detection method comprises a step of incubating a sample with an oligonucleotide probe. In some embodiments, a sample comprises one or more oligonucleotides.

In some embodiments, hybridization or incubation comprises annealing. In some embodiments, such hybridization or incubation comprising annealing occurs at one or more annealing temperatures. In some such embodiments, an annealing temperature is 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, or 65° C.

In some embodiments, a sample comprising the oligonucleotide is subject to one or more digestion steps prior to incubating with an oligonucleotide probe to cleave and remove the peptide from the oligonucleotide and/or the sample. In some embodiments, the one or more digestion steps comprises a nuclease (e.g., trypsin). In some embodiments, one or more digestion steps comprises more than one nuclease. In some embodiments, a nuclease is a nuclease that cleaves a bond between a peptide and an oligonucleotide.

In some embodiments, a detection method comprises a step of incubating a sample/oligonucleotide probe mixture with a plate coated with a capture agent. In some such embodiments, the capture agent binds the oligonucleotide probe (e.g., via a covalently linked label) thereby causing the oligonucleotide probe and hybridized oligonucleotides to become associated with the plate.

In some embodiments, a detection method comprises a step of washing a plate after a sample/oligonucleotide probe mixture is incubated with a plate coated with a capture agent. In some such embodiments, washing removes oligonucleotide probes and/or oligonucleotides that are not associated with the plate.

In some embodiments, a detection method comprises incubating a plate to which one or more oligonucleotide probes is associated (and which oligonucleotide probes may or may not each be hybridized to an oligonucleotide) with one or more single strand-specific nucleases. In some embodiments, the single strand-specific nuclease is micrococcal nuclease. In some embodiments, the single strand-specific nuclease is mung bean nuclease. In some embodiments, both micrococcal and mung bean nuclease are used in a detection method as described herein.

In some such embodiments, the incubation comprises incubation with micrococcal nuclease, followed by incubation with mung bean nuclease. In some such embodiments, a step of washing the plate is included between incubation with micrococcal and mung bean nuclease. In some embodiments, the incubation comprises only micrococcal nuclease. In some embodiments, the incubation comprises only mung bean nuclease. In embodiments comprising the same nuclease (e.g., mung bean nuclease), an optional wash step may be included between a first and second incubation (e.g., with both incubations comprising the same nuclease).

In some embodiments, micrococcal nuclease and/or mung bean nuclease preferentially cleave excess single-stranded oligonucleotide probes. In some embodiments, mung bean nuclease has a higher specificity for such cleavage than micrococcal nuclease.

In some embodiments, a substrate that has been incubated with one or more single strand-specific nucleases is further incubated with a detection agent. In some embodiments, if a detection agent is incubated with one or more components, a detectable signal is produced. In some such embodiments, the detection agent interacts with an oligonucleotide probe (e.g., with a covalently linked label) to produce a detectable signal. In some embodiments, a detectable signal is only produced when an oligonucleotide probe is hybridized to an oligonucleotide.

In some embodiments, the detectable signal is detected. In some embodiments, the detection is colorimetric. In some embodiments, the detection is non-colorimetric. In some such embodiments, the detection is or comprises measurement of optical density. In some embodiments, detection is determined using fluorescence detection using set excitation and/or emission wavelengths.

In some embodiments, a detectable signal is produced using a probe specific targeting moiety. In some embodiments, a probe specific targeting moiety comprises a conjugated enzymatic agent (e.g., an anti-body enzyme conjugate). In some embodiments, a probe specific targeting moiety conjugate comprises an antibody conjugate. In some embodiments, an antibody conjugate comprises an anti-digoxigenin antibody conjugated with an enzymatic moiety (e.g., alkaline phosphatase). In some embodiments, a suitable substrate (e.g., a suitable alkaline phosphatase substrate e.g., 2′-[2-benzothiazoyl]-6′-hydroxybenzothiazole phosphate [BBTP]) is added to an assay plate, and the subsequent reaction is permitted to incubate for a suitable period of time to allow production of a fluorescence signal. In some embodiments, a suitable period of time to allow production of a fluorescence signal is less than two hours, less than 90 minutes, less than 60 minutes, less than 45 minutes, or less than 30 minutes. In some embodiments, a suitable period of time to allow production of a fluorescence signal is apprately 30 minutes. In some embodiments, fluorescence excitation occurs at a range of 400-500 nm, at a range of 410-490 nm, at a range of 420-480 nm, at a range of 430-470 nm, at a range of 440-460 nm, a range of 440-450 nm, or approximately 444 nm. In some embodiments, fluorescence emission detection occurs at a range of 500-600 nm, at a range of 510-590 nm, at a range of 520-580 nm, at a range of 530-570 nm, at a range of 540-560 nm, at a range of 550-560 nm, or at approximately 555 nm. One of skill in the art will recognize that alternative detection agents and associated signal detection methods may be suitable for use in an assay as described herein.

In some embodiments, an assay as described herein is capable of detecting the presence and/or quantity of an oligonucleotide in a given sample at a lower level of detection than other existing or previously used assays. In some embodiments, a level of oligonucleotide is below a level of detection. In some embodiments, a level of detected oligonucleotide detects all oligonucleotide comprised within a given sample. In some embodiments, a level of detected oligonucleotide is equal to or lesser than an amount of oligonucleotide administered to a subject. In some such embodiments, the equal or lesser amount is due to dilution in the subject and not due to a level of detection. That is, in some embodiments, an assay as described herein, comprising an oligonucleotide probe as described herein, is capable of accurately quantifying an amount of oligonucleotide present in a given sample.

### Oligonucleotide Designs

In some embodiments, the present disclosure provides one or more oligonucleotide probes for use in detection of one or more oligonucleotides. In some embodiments, one or more oligonucleotide probes is/are or comprise one or more types of nucleic acids (e.g., locked nucleic acids and deoxyribonucleic acids). In some such embodiments, use of oligonucleotide probes of the present disclosure in methods described herein result in improved detection of oligonucleotides as compared to previously available detection methods. For example, use of methods as described herein show improved accuracy, sensitivity, and/or reproducibility in detection of one or more oligonucleotides in a given sample as compared to previously available methods for detection of oligonucleotides in a sample.

Oligonucleotide Probes

In some embodiments, an oligonucleotide probe is or comprises one or more nucleic acids. In some embodiments, an oligonucleotide probe is or comprises one or more modified nucleic acids. For example, in some embodiments, an oligonucleotide probe is or comprises PTO and DNA nucleic acids. In some embodiments, an oligonucleotide probe is or comprises LNA and DNA nucleic acids. In some embodiments an oligonucleotide probe comprising, e.g., PTO and DNA or, e.g., LNA and DNA is arranged such that PTO or LNA residues are present on 3′ and 5′ ends of an oligonucleotide probe and flank a middle region comprising DNA. In some embodiments, an LNA/DNA oligonucleotide comprises a biotin label on the 3′ end and/or a digoxigenin label on the 5′ end.

Oligonucleotides

In some embodiments, an oligonucleotide is or comprises a PMO. In some embodiments an oligonucleotide is linked to a peptide (e.g., to produce a P-PMO). In some embodiments, a sample comprising the oligonucleotide is subject to one or more digestion steps prior to incubating with the oligonucleotide probe to cleave and remove the peptide from the oligonucleotide and/or the sample. In some embodiments, an oligonucleotide is between about 10-100 nucleotides in length. In some embodiments, an oligonucleotide is between about 12-85 nucleotides in length. In some embodiments, an oligonucleotide is between about 15 and 60 oligonucleotides in length. In some embodiments, an oligonucleotide is between about 20 and 50 oligonucleotides in length. In some embodiments, an oligonucleotide is between about 25-35 oligonucleotides in length.

### Methods of Use of Oligonucleotide Detection Assays

Prior to the insight provided by the present disclosure, related assays to detect oligonucleotides were limited in reproducibility, accuracy, and/or sensitivity. The methods of the present disclosure are not limited to detection of any particular type of oligonucleotide detection in any particular setting. For example, in some embodiments, the methods of the present disclosure may be used to monitor patient response to delivery of an oligonucleotide as described herein. In some embodiments, the methods of the present disclosure may be used in evaluation of efficacy and toxicity of candidate oligonucleotides during research and development procedures.

The present disclosure provides methods of detection that achieve unprecedented sensitivity and also display reproducibility and accuracy. Such methods can be used for any portion of oligonucleotide development and use, e.g., from pre-clinical candidate screening into in vivo monitoring of levels of oligonucleotides in one or more samples from a patient. In some embodiments, such in vivo monitoring could include, for example, overall serum level monitoring, or, monitoring of levels in a particular sample from a particular organ or tissue (e.g., muscle) to determine an amount of oligonucleotide reaching that organ or tissue. In some embodiments, such in vivo monitoring could include, for example, detecting levels in a sample from an organ or tissue (e.g., kidney, muscle, etc.) to monitor oligonucleotide load or concentration in a particular organ or tissue. In some embodiments, the organ or tissue is one or more of blood, plasma, serum, skin, lungs, heart, gastrocnemius, biceps, tibialis anterior (TA), quadriceps, diaphragm, central nervous system tissue (e.g., brain, spinal cord), or a combination thereof.

In some embodiments, the present disclosure utilizes improved oligonucleotide probes to detect oligonucleotides which have been administered to a subject in need thereof. For example, in some embodiments, a subject in need thereof is administered one or more oligonucleotides. In some such embodiments, one or more oligonucleotide probes may be designed and used to detect the one or more oligonucleotides that was and/or is being administered to a subject.

In some embodiments, the methods of the present disclosure comprise administering and/or detecting an oligonucleotide or candidate oligonucleotide. In some embodiments, the methods of the present disclosure comprise administering and/or detecting more than one type of oligonucleotide.

In some embodiments, detection of an oligonucleotide occurs after administration. In some embodiments, detection occurs about 0.25, 0.5, 0.75, 1, 2, 3, 4, 5, 6, 7, 8, 9, 10, 11, 12, 13, 14, 15, 16, 17, 18, 19, 20, 21, 22, 23, 24, 48, or 72 hours or more after administration. In some embodiments, detection occurs about 1, 2, 3, 4, 5, 6, 7, 8, 9 10, 11, 12, 13, 14, 15, 16, 17, 18, 19, 20 or more weeks after administration. In some embodiments, detection is performed at regular intervals (e.g., every 1, 2, 3, 4, 5, 6, 7, 8, 9, or 10 or more weeks). In some embodiments, an administration is the first administration of an oligonucleotide. In some embodiments, an administration is the most recent administration, but not necessarily first administration, of an oligonucleotide.

In some embodiments, a sample comprises serum and/or tissue from a subject to whom an oligonucleotide was administered. In some embodiments, a sample comprises serum and/or tissue from a subject to whom an oligonucleotide was not administered (e.g., a control subject who received no oligonucleotide or a placebo oligonucleotide).

### Duchenne Muscular Dystrophy

Duchenne muscular dystrophy (DMD) is a recessive X-linked form of muscular dystrophy, which results in muscle degeneration/wasting and, at present, ultimately leads to death. DMD affects approximately 1:3500 males. DMD is caused by one or more changes in the dystrophin gene, which leads to disrupted reading frames and loss of some, most, or all functional dystrophin protein expression and/or production. Dystrophin is an important structural component within muscle tissue and alteration or absence of dystrophin results in abnormal membrane function in muscle cells.

The dystrophin gene is located on the X chromosome, meaning males (XY sex chromosomes) are hemizygous for the gene and typically present and progress with more severe symptoms than do females. Symptoms are known to include early physical disability and mortality. In some cases, male symptom development trajectory may include loss of mobility by adolescence, compromised respiratory and cardiac function by late teens, and death in early adulthood. Whereas, females (XX sex chromosomes) carrying a heterozygous dystrophin mutation typically present with a milder phenotype. Changes in the dystrophin gene that preserve the reading frame result in the milder, non-life threatening Becker muscular dystrophy (BMD).

The dystrophin gene is large, with 79 exons, and the most common genetic changes in DMD include genomic deletion of one or more exons. Most commonly, such deletions involve areas around or within exons 44 to 55 and/or at the 5′ end of the gene. At present, there is no known cure for DMD, but in addition to historical use of corticosteroids, certain antisense-oligonucleotide-based therapeutics have been approved for use in patients with DMD.

Treatment based on exon skipping induced by oligonucleotides works via targeted exon skipping during the splicing process that produces functional dystrophin mRNA which is translated into functional protein. This process uses complex, multi-particle cellular machinery to bring adjacent exon-intron junctions in pre-mRNA into close proximity with one another, permitting the cleavage of the phosphodiester bonds found at the ends of introns and ultimately resulting in spicing together of exons. As is now well known in the art, use of certain antisense oligonucleotides (ASOs) can facilitate the bypassing or removal of certain errors in a transcribed pre-mRNA molecule as it is spliced to from a mature coding mRNA transcript. For example, skipping out-of-frame mutations of the dystrophin gene can result in restoration of the reading frame and production of a functional (albeit truncated) dystrophin protein.

There are various types of oligonucleotides based on DNA and/or RNA backbones or analogs thereof, as well as oligonucleotide conjugates to various types of peptides for improved cellular penetration. Nonetheless, there remains an unmet need in ability to accurately and sensitively detect such oligonucleotides in samples from patients. If treatments are going to involve use of oligonucleotides, it is very important that accurate, sensitive, and reproducible detection of those oligonucleotides be made possible. The technologies provided by the present disclosure offer accurate, sensitive, and reproducible detection of such oligonucleotides.

The disclosure is further illustrated by the following example. This example is provided for illustrative purposes only. It is not to be construed as limiting the scope or content of the disclosure in any way.

### Example

The present disclosure exemplifies methods of detecting one or more oligonucleotides in a sample.

### Example 1: Oligonucleotide Detection Assay

The present example describes an exemplary assay for detection of oligonucleotides in a sample. The assay of the present disclosure surprisingly and significantly improves detection capabilities above and beyond any currently available assays used to detect oligonucleotides. The assay described herein shows improved accuracy, sensitivity and reproducibility as compared to other available detection methods.

In the present example, oligonucleotides were initially provided in a composition comprising a peptide and oligonucleotide, as peptide phosphorodiamidate morpholino oligonucleotides (P-PMOs).

### Probe Sequences and Designs

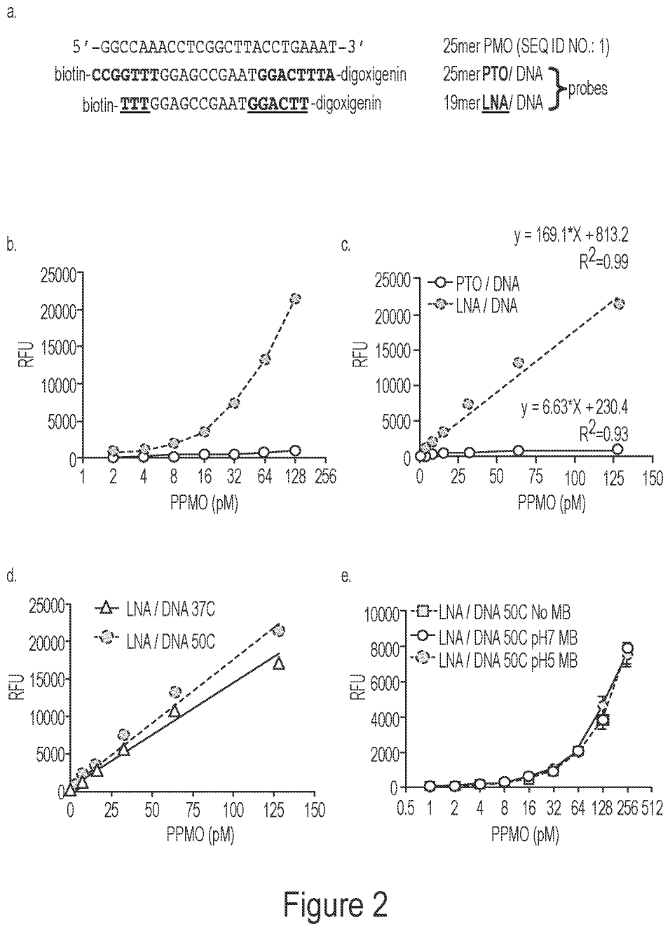

The present assay used locked nucleic acid (LNA)/DNA oligonucleotide probes for detection of oligonucleotides. LNA/DNA oligonucleotide probes were designed with LNA bases at 5′ and 3′ ends of each oligonucleotide probe and compared to phosphorothioate (PTO)/DNA oligonucleotide probes (see, e.g., Burki et al., 2015, Nucleic Acid Therapeutics, 25(5), pp. 275-84, for description of PTO/DNA probes), as in FIG. 2.

Exemplary Sequence and Oligonucleotides

In the present example, the oligonucleotides were derived from peptide-conjugated antisense oligonucleotides and antisense oligonucleotides that are not conjugated to a peptide. Specifically, as shown in FIG. 2a, a 25-mer PMO antisense sequence for mouse dystrophin exon-23 (M23D) with sequence (5′-GGCCAAACCTCGGCTTACCTGAAAT-3′; SEQ ID NO: 1) was designed in-house and ordered from a commercial oligonucleotide supplier. For peptide-conjugated oligonucleotides, a peptide-PMO (“P-PMO”) was synthesized by conjugation of the M23D PMO with one of two cell-penetrating peptides (peptide A or B, to create P-PMO A and/or P-PMO B), each of which contains 18 amino acids.

Oligonucleotide Probes

The complementary oligonucleotide probes for each of P-PMO A and P-PMO B were designed and ordered from Exiqon (LNA/DNA probe; Woburn, Mass.) or IDT (PTO/DNA probe; Coralville, Iowa). As shown in FIG. 2a, the oligonucleotide probes for M23D PMO were a “truncated” 19-mer LNA/DNA probe (3′-TTTGGAGCCGAATGGACTT-5′; SEQ ID NO: 2; LNA bases highlighted in bold and underlined in FIG. 2a) and a “full-length” 25-mer PTO/DNA probe (3′-CCGGTTTGGAGCCGAATGGACTTTA-5′; SEQ ID NO: 3; PTO bases highlighted in bold in FIG. 2a). Both probes were tested during assay development, and after finalizing assay methodology, the truncated probe was used for all subsequent assays (e.g., quantification assays, e.g., in vivo detection and quantification of PMO levels, etc.). Both oligonucleotide probes were dual-labeled with biotin at the 3′-end and digoxigenin at the 5′-end. The lyophilized oligonucleotide probes were suspended in nuclease free water at 10 μM to prepare a stock solution. PMOs and P-PMOs were suspended in distilled water at 10 mg/ml stock. The probes were aliquoted and stored at −20° C. PMOs and P-PMOs were aliquoted and stored at −70° C. All probes, PMOs, and P-PMOs were heated at 65° C. for 15 min and briefly vortexed before commencing the ELISA.

### ELISA-Based Detection Assay

PMO or P-PMO serial dilutions for standard curves and quality controls in 5.0% mouse serum, and 5.0 mg/ml or 1.0 mg/ml tissue homogenates were made using 1×TE buffer supplemented with 0.1% v/v Triton X-100 with a starting concentration of 51.2 nM PMO/P-PMO. Serum samples were diluted 20-fold, and tissues were homogenized and diluted to 5.0 mg/ml or 1.0 mg/ml tissue homogenates with the same buffer. 200 μL of calibrator solution, quality control, and/or sample were treated with 20 μL of 40 mg/ml trypsin (purchased as lyophilized powder from Sigma-Aldrich) at 37° C. overnight in a deep 1.0-mL 96-well plate on thermomixer C (600 rpm) to cleave the peptide component and release the PMO. 40 μL of 2.5 nM oligonucleotide probe in hybridization buffer (with 0.1% serum) was added to each well. The plate was then sealed, incubated at 65° C. for 15 minutes (600 rpm), and allowed to cool at room temperature for 15 minutes. The plate was then incubated at 50° C. for 30 minutes (500 rpm) to allow the oligonucleotide probe to hybridize with the PMO. One hundred and fifty microliters of the hybridized solution was then transferred to the NeutrAvidin-coated plates (pre-washed using a wash buffer: 50 mM Tris-HCl, 150 mM sodium chloride, pH 7.6, 0.1% v/v Tween-20—the same wash buffer was used for all subsequent washing steps) and incubated at 37° C. for 30 minutes to allow the biotin labeled probes to bind to the NeutrAvidin-coated plate. The plate was then washed three times and 150 μL of 0.2 U/μl micrococcal nuclease (in 50 mM Tris-HCL; pH 8.2, 200 mM NaCl, 5 mM CaCl, and 0.1 mg/mL bovine serum albumin) was added to each well and incubated at 37° C. for 1.5 h (150 rpm). The plate was then washed three times and 120 μL of 0.3 U/μL mung bean nuclease (in 30 mM NaCl; pH 8.2, 50 mM sodium acetate, 1 mM ZnSO4; pH 5.0) was added to each well and incubated at 37° C. for 1.5 h (150 rpm). The plate was washed three times and 150 μL of anti-digoxigenin antibody conjugated to alkaline phosphatase was added at 1:5,000 dilution in SuperBlock (TBS) Blocking Buffer with 0.25% v/v Tween-20, incubated at 37° C. for 30 minutes followed by washing three times. Then, 125 μL of AttoPhos substrate was added to each well, and the plates were sealed in aluminum foil and incubated at 37° C. for 30 minutes (150 rpm). The intensity of fluorescence at 444 nm excitation and 555 nm emission was measured by a Molecular Service SpectraMax MT5 microplate reader.

### PTO/DNA Vs LNA/DNA Oligonucleotide Probes

When compared in the ELISA-based detection assay of the present example, PTO/DNA oligonucleotide probes resulted in a minimal fluorescent signal when oligonucleotide was present at less than 64 pM (See FIGS. 2b and 2c). Surprisingly, and in contrast, LNA-containing oligonucleotide probes resulted in significantly higher fluorescence signal than PTO/DNA oligonucleotide probes, including at low oligonucleotide concentrations. Signal-to-noise ratios and limits of quantitation were also improved using LNA-containing oligonucleotide probes as compared to PTO-containing oligonucleotide probes. For example, a lower limit of quantification (“LLOQ”) for the PTO/DNA oligonucleotide probes was less than 8 pM (S:N set to less than 2; see Table 1 below), consistent with previous reports for this type of probe (see, e.g., Burki et al., 2015, Nucleic Acid Therapeutics, 25(5), pp. 275-84). In contrast, 2 pM concentration of oligonucleotides was detected using LNA/DNA oligonucleotide probes and a robust S:N was observed (S:N=5.2).

### Annealing and Digestion

The present example also describes certain procedural modifications, relative to existing assays, which surprisingly and significantly improved accuracy, reproducibility, and lowered limits of detection of oligonucleotide in a sample to picomolar concentrations.

In the present Example, both annealing temperature and digestion conditions were varied and analyzed (See FIGS. 2d and 2e). When oligonucleotide annealing temperature was increased relative to that of existing assays, improvement in signal was observed (FIG. 2d). Signal intensity was unchanged in the presence or absence of a second digestion step using mung bean (MB) nuclease (FIG. 2e). However S:N was improved when residual single-stranded oligonucleotide was digested in the presence of mung bean (MB) nuclease, in particular at pH 5 (see Table 2 below).

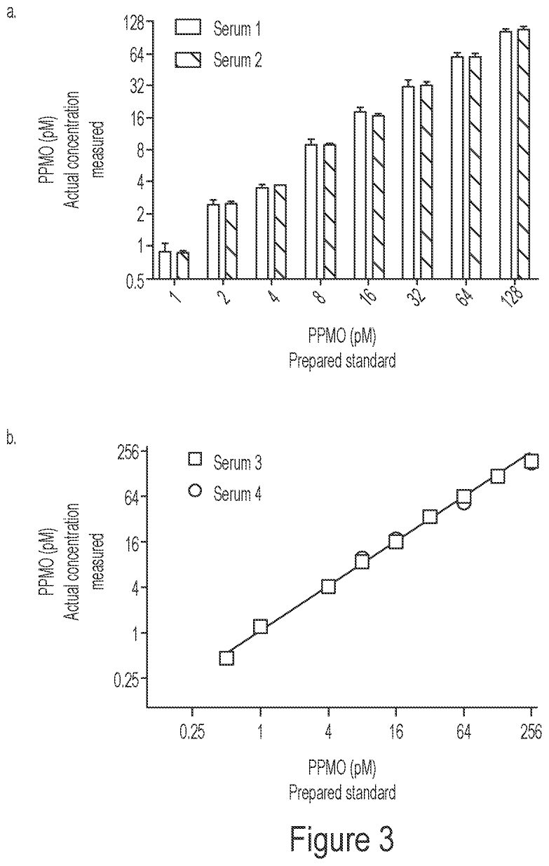

The detection assay was qualified in mouse serum. Accuracy and reproducibility of standard curves were analyzed using at least eight prepared standards (calibrators) made by spiking in known concentrations of PMO diluted in 5% mouse serum. FIGS. 3a and 3b show reproducibility and accuracy of the detection assay with P-PMO standards prepared in four different sera and run in two independent assays. Quality control samples were considered acceptable if back-calculated concentrations had a CV below 25%. Tables 3 and 4 show accuracy of the assay in different serum preparations using different P-PMOs. CVs were generally below 20%, except for detection of P-PMO A at the LLOQ of 1 pM (29.7%). Inter-batch variability was below 20% between assays, with the exception of P-PMO A at 1 pM (20.3%).

The detection assay described in this Example was also validated utilizing several mouse tissues. The assay detects and measures levels of PMOs after a trypsin digestion, which digestion cleaves the peptide (“P”) from the PMO. P-PMO levels are inferred through detection of PMO levels as determined by an assay such as that described herein. It is known that PMO uptake in tissue is poor, and that peptide conjugation to create P-PMOs facilitates uptake into tissue; thus it is inferred that the majority of PMO detected in tissues is due to uptake mediated by P-PMO. Table 5 shows mean detection values obtained at the LLOQ across these tissues listed in the table. Variability was below 25% for all tissues analyzed, across assays and independent of which P-PMO was used.

### Dynamic Detection Using In Vivo Samples

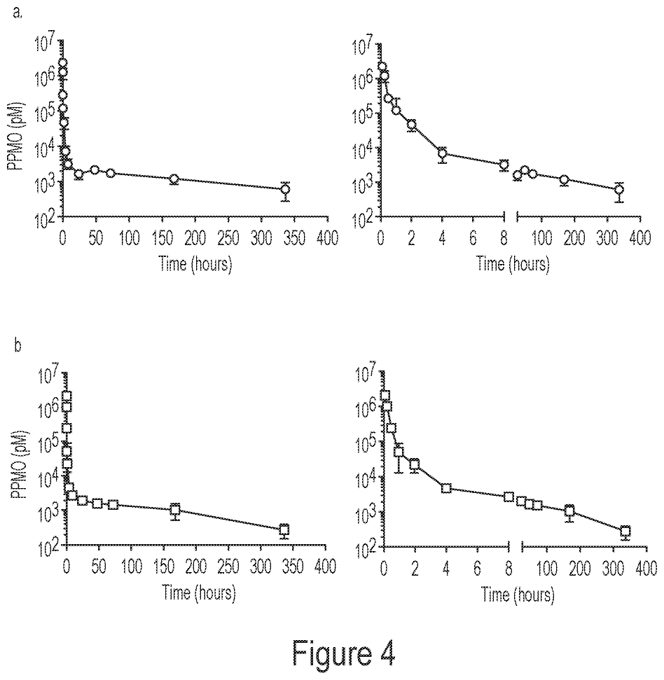

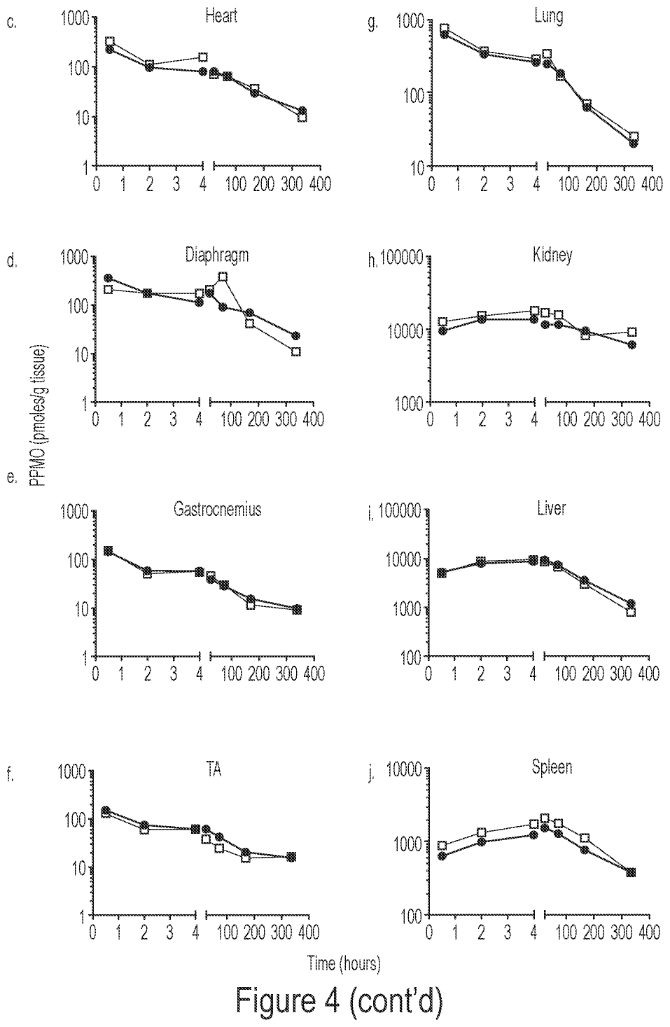

Serum levels of P-PMO A (by measurement of PMO) were measured in CD1 mice over a two week time period. P-PMO levels were determined using oligonucleotide probes to detect levels of PMOs conjugated to its delivery peptide. FIG. 4a shows initial serum concentration of 2.3 μM (−/+0.2 μM) after five minutes; left and right panels show the same data, but the right panel has a divided x-axis).

Detected P-PMO A concentrations dropped rapidly over the first eight hours at which time the concentration detected was 3.3 nM (−/+1.1 nM). P-PMO A serum levels remained relatively stable for one week, at which time concentration detected was 1.2 nM (−/+0.38 nM). After two weeks, concentration detected was 609 pM (−/+327 pM). Similar data were observed when analyzing P-PMO B (FIG. 4b).

As shown in FIGS. 4c, 4d, 4e, 4f, 4g, 4h, 4i, and 4j, P-PMO levels in several tissues were also determined. In target tissues (heart, diaphragm, gastrocnemius, tibialis anterior (TA)) there was an initial drop in detectable P-PMO concentration (0.5 to 2 hour time points). Thereafter P-PMO concentration detected in these tissues remained relatively constant for at least three days. P-PMO levels remained detectable (and quantifiable) throughout the study. In first pass organs (e.g., lung, kidney), higher concentrations of P-PMOs were detected. Over time and after three days post-dose, P-PMO concentrations decreased steadily. Clearance in the kidney was delayed, consistent with a known class effect for oligonucleotides (e.g., ASOs). Both P-PMO A and P-PMO B showed similar drug levels in serum and all tissues at each time point analyzed.

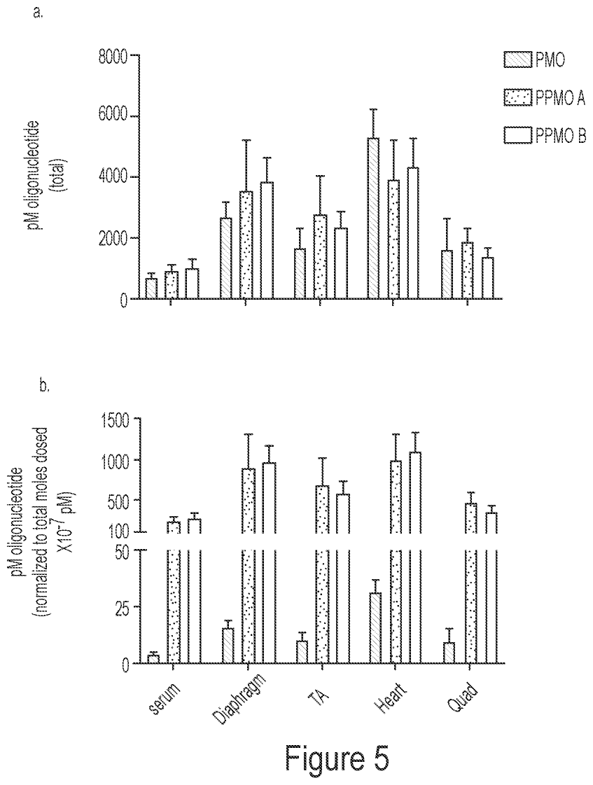

In order to test the persistence of oligonucleotides, serum and various tissues were also analyzed two weeks post-last dose. Serum levels for both PMO and P-PMO treated mice were measured in the 1 nM range. At this time point the overall oligonucleotide levels in the tissues analyzed were also similar (FIG. 5a).

To account for differences in doses administered, drug levels (e.g., P-PMO A and corresponding PMO not conjugated to a peptide) were normalized to total number of moles dosed. FIG. 5b demonstrates that though number of moles of P-PMO dosed was 43-fold less than number of moles of PMO not conjugated to a peptide, P-PMO was detected at much higher concentrations in serum and tissue than PMO not conjugated to a peptide. Furthermore, that oligonucleotide concentration was at least 60-fold higher in several target tissues following P-PMO treatment as compared to treatment with a PMO not conjugated to a peptide.

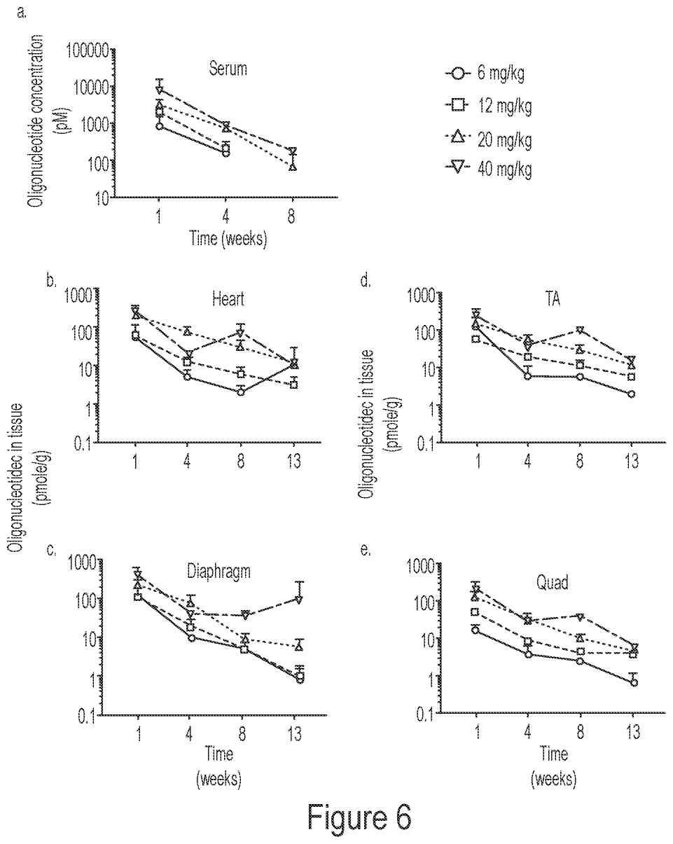

These data demonstrate the unexpected sensitivity of the present assay to detect an oligonucleotide for at least two weeks post-last dose. Assessment of oligonucleotide levels after even longer time periods post-administration could be clinically useful, for example, if oligonucleotide levels could be correlated with efficacy. Thus, to determine sensitivity of the present detection assay described in this Example, PMO levels in serum and several muscle tissues were monitored for 13 weeks post-drug administration (e.g., post-oligonucleotide administration, e.g., post-P-PMO administration). FIG. 6a shows that P-PMO B was detectable in serum of mice dosed at 6 or 12 mg/kg for up to four weeks post-administration, but was not detectable by eight weeks post-administration within the limits of detection of the assay. At doses of 20 and 40 mg/kg P-PMO B was still detectable in serum at eight weeks post-administration, but was no longer detectable by 13 weeks. Analysis of certain exemplary skeletal muscles (6b heart, 6c diaphragm, 6d TA, and 6e quadriceps) showed detectable oligonucleotide in all tissues for up to 13 weeks post-administration (the duration of the study).

An advantage of the presently described assay relative to any other available assay is demonstrated by increased sensitivity that allows reliable and accurate quantification of P-PMO levels at 13 weeks post-administration using the method described herein for detection at levels that were previously undetectable by other assays. Mean P-PMO concentrations detected at 13 weeks post-administration were between: 3-10 pmole/g in heart; 1-90 pmole/g in diaphragm; 1.8-15.8 pmole/g in TA; and 0.6-5.7 pmole/g in quad.

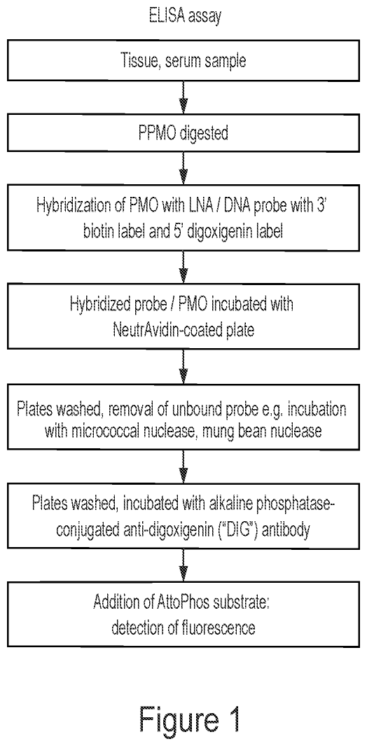

Importantly, the very low concentrations of oligonucleotide detected in the assay described herein were neither detectable nor quantifiable at such low levels in other previously described assays (Burki et al., 2015, Nucleic Acid Therapeutics, 25(5), pp. 275-84), demonstrating the superiority of the presently disclosed assay. Also, when data from two different in vivo studies (as represented by data in FIGS. 4 and 6) were compared to data obtained using the ELISA assay with in vitro samples (e.g., such as in FIG. 1), results were similar, demonstrating the reproducibility of the presently disclosed assay(s). Week 1 data for heart, TA and diaphragm were compared (between studies represented in FIGS. 4 and 6 at dose of 6 mg/kg), and although there were some individual outliers (two each for TA and diaphragm), data (excluding outliers) were overall comparable and reproducible between studies. Levels from two different studies were as follows at week 1 for each tissue assayed: heart, 36 vs 55 pmoles/g; diaphragm, 42 vs 43 pmoles/g; TA, 15 vs 23 pmoles/g, as represented in data shown in FIGS. 4 and 6, respectively.

### Tables

TABLE 1
Fluorescence Intensities (RFU) and signal to noise (S:N) ratios
obtained following incubation of PTO/DNA or LNA/DNA
probes with P-PMO.

| | | PPMO concentration (pM) | | | | | | | |
|---|---|---|---|---|---|---|---|---|---|
| | | 0 | 3 | 4 | 8 | 16 | 32 | 64 | 128 |
| PTO/ | RFU | 115 | 172 | 222 | 342 | 413 | 520 | 734 | 1009 |
| DNA | S:N | | 1.5 | 1.9 | 3.0 | 3.6 | 4.5 | 6.4 | 8.7 |
| LNA/ | RFU | 144 | 746 | 1081 | 1977 | 3415 | 7450 | 13261 | 21382 |
| DNA | S:N | | 5.2 | 7.5 | 13.7 | 23.7 | 51.8 | 92.2 | 148.6 |

TABLE 2
Signal to noise (S:N) ratios obtained following incubation of LNA/DNA
probes with P-PMO in the presence of absence of mung bean (MB)
nuclease at different temperatures

| | PPMO concentration (pM) | | | | | | | | |
|---|---|---|---|---|---|---|---|---|---|
| | 1 | 2 | 4 | 8 | 16 | 32 | 64 | 128 | 256 |
| No MBN | 1.9 | 2.6 | 6.4 | 8.4 | 15.6 | 31.3 | 68.4 | 128.6 | 252.9 |
| +MBN pH7 | 2.5 | 3.7 | 6.7 | 11.5 | 20.1 | 33.9 | 69.8 | 133.1 | 262.0 |
| +MBN pH5* | 3.6 | 5.7 | 8.4 | 15.8 | 31.4 | 60.5 | 112.0 | 240.0 | 400.1 |

(asterisk(*) indicates conditions used in subsequent assays).

TABLE 3
Qualification of hybridization assay. Accuracy of assay determined
using known concentrations of P-PMO A added to serum.

| | | PPMO concentration (pM) | | | | |
|---|---|---|---|---|---|---|
| | | LLOQ | Low | Mid | High | ULOQ |
| | | prepared concentration | | | | |
| | | 1.0 | 3.0 | 12.0 | 100 | 128 |
| Serum 1 | | | | | | |
| Measured | 1 | 0.867 | 3.31 | 12.9 | 86.3 | 111 |
| concentration | 2 | 0.895 | 3.34 | 12.6 | 111 | 115 |
| | 3 | 0.915 | 3.76 | 12.0 | 84.9 | 140 |
| Mean | | 0.892 | 3.47 | 12.5 | 94.1 | 122 |
| % CV | | 2.7 | 7.3 | 3.7 | 15.7 | 12.9 |
| % bias | | −10.8 | 15.8 | 4.1 | −5.9 | −4.8 |
| Serum 2 | | | | | | |
| Measured | 1 | 0.512 | 3.13 | 13.5 | 90.0 | 138 |
| concentration | 2 | 0.946 | 3.08 | 12.8 | 88.4 | 119 |
| | 3 | 0.727 | 3.24 | 13.0 | 90.2 | 111 |
| Mean | | 0.728 | 3.15 | 13.1 | 89.5 | 123 |
| % CV | | 29.7 | 2.7 | 2.7 | 1.1 | 11.3 |
| % bias | | −27.2 | 5.0 | 9.1 | −10.5 | −3.8 |
| Inter-batch | | | | | | |
| statistics | | | | | | |
| Mean | | 0.810 | 3.31 | 12.8 | 91.8 | 122 |
| % CV | | 20.3 | 7.4 | 3.8 | 10.6 | 10.8 |
| % bias | | −19.0 | 10.4 | 6.6 | −8.2 | −4.3 |
| n | | 6 | 6 | 6 | 6 | 6 |

LLOQ = Lower limit of quantification;
ULOQ = Upper limit of quantification.

TABLE 4
Qualification of hybridization assay. Accuracy of assay determined
using known concentrations of P-PMO B added to serum.

| | | PPMO concentration (pM) | | | | |
|---|---|---|---|---|---|---|
| | | LLOQ | Low | Mid | High | ULOQ |
| | | prepared concentration | | | | |
| | | 1.0 | 3.0 | 12.0 | 100 | 128 |
| Serum 1 | | | | | | |
| Measured | 1 | 0.706 | 2.92 | 13.0 | 86.4 | 102 |
| concentration | 2 | 0.865 | 3.04 | 12.5 | 84.5 | 100 |
| | 3 | 0.967 | 3.41 | 12.7 | 84.5 | 106 |
| Mean | | 0.846 | 3.12 | 12.7 | 85.1 | 103 |
| % CV | | 15.6 | 8.2 | 1.98 | 1.25 | 3.21 |
| % bias | | −15.4 | 4.1 | 6.0 | −14.9 | −19.9 |
| Serum 2 | | | | | | |
| Measured | 1 | 0.815 | 3.21 | 13.3 | 81.9 | 104 |
| concentration | 2 | 0.853 | 3.43 | 13.5 | 85.3 | 105 |
| | 3 | 0.970 | 3.86 | 14.4 | 87.9 | 105 |
| Mean | | 0.879 | 3.5 | 13.8 | 85.0 | 105 |
| % CV | | 9.21 | 9.42 | 4.25 | 3.53 | 0.424 |
| % bias | | −12.1 | 16.7 | 14.7 | −11.7 | −18.3 |
| Inter-batch | | | | | | |
| statistics | | | | | | |
| Mean | | 0.863 | 3.31 | 13.2 | 85.1 | 104 |
| % CV | | 11.5 | 10.1 | 5.27 | 37 | 2.31 |
| % bias | | −13.7 | 10.4 | 10.3 | −14.9 | −19.1 |
| n | | 6 | 6 | 6 | 6 | 6 |

LLOQ = Lower limit of quantification;
ULOQ =Upper limit of quantification.

TABLE 5
Accuracy and variability of hybridization
assay across murine tissues

| | | Mean | |
|---|---|---|---|
| | | detection at | |
| | | LLOQ (1 pM) | % CV |
| PPMO | Tissue | Inter-batch statistic | |
| A | Serum | 0.810 | 20.3 |
| | Heart | 0.897 | 9.6 |
| | Spleen | 0.960 | 12.5 |
| | Lung | 1.14 | 14.4 |
| | Kidney | 0.820 | 12.5 |
| | Liver | 0.956 | 22.7 |
| | Diaphragm | 0.859 | 19.3 |
| | Gastrocnemius | 1.14 | 14.4 |
| | Tibialis Anterior | 1.14 | 14.4 |
| B | Serum | 0.863 | 11.5 |
| | Heart | 0.858 | 19.2 |
| | Spleen | 0.818 | 23.3 |
| | Lung | 1.14 | 13.2 |
| | Kidney | 0.926 | 9.9 |
| | Liver | 0.929 | 9.3 |
| | Diaphragm | 0.858 | 11.7 |
| | Gastrocnemius | 1.14 | 13.2 |
| | Tibialis Anterior | 1.14 | 13.2 |

### EQUIVALENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

We claim:

1. A method comprising steps of:
(a) incubating a sample comprising oligonucleotides with an oligonucleotide probe, wherein the oligonucleotide probe hybridizes with the oligonucleotides in the sample;
(b) incubating the product of step (a) with a substrate coated with a capture agent, wherein the capture agent binds the oligonucleotide probe thereby causing the excess oligonucleotide probe and hybridized oligonucleotides to become associated with the substrate, and optionally washing the plate after the hybridized probes become associated with the substrate;
(c) incubating the substrate after step (b) with two or more different single-strand-specific nucleases that have a different specificity for the substrate, wherein the substrate is optionally washed with one or more wash solutions before, during or after step (c), and the oligonucleotide probe and hybridized oligonucleotides remain associated with the substrate during the washing step or steps;
(d) incubating the substrate after step (c) with a detection agent, wherein the detection agent interacts with the oligonucleotide probe to produce a detectable signal; and
(e) detecting the detectable signal.

2. A method comprising steps of:
(a) incubating a sample comprising phosphorodiamidate morpholino oligonucleotides (PMOs) with an oligonucleotide probe, wherein the oligonucleotide probe comprises one or more locked nucleic acid (LNA) residues and hybridizes with PMOs in the sample;
(b) incubating the product of step (a) with a substrate coated with a capture agent, wherein the capture agent binds the oligonucleotide probe thereby causing the oligonucleotide probe and hybridized PMOs to become associated with the substrate;
(c) incubating the substrate after step (b) with one or more single-strand-specific nucleases, wherein the substrate is optionally washed before, during or after step (c) and the oligonucleotide probe and hybridized PMOs remain associated with the substrate during the washing step or steps;
(d) incubating the substrate after step (c) with a detection agent, wherein the detection agent interacts with the oligonucleotide probe to produce a detectable signal; and
(e) detecting the detectable signal.

3. The method of claim 1, wherein step (c) comprises incubating the substrate with micrococcal nuclease and mung bean nuclease.

4. The method of claim 3, wherein step (c) comprises incubating the substrate with the micrococcal nuclease and then incubating the substrate with the mung bean nuclease.

5. The method of claim 4, wherein the substrate is washed after it has been incubated with the micrococcal nuclease and before it is incubated with the mung bean nuclease.

6. The method of claim 1, wherein the sample comprises phosphorodiamidate morpholino oligonucleotides (PMOs).

7. The method of claim 1, wherein the oligonucleotide probe comprises one or more locked nucleic acids (LNA) residues.

8. The method of any of the preceding claims, wherein the oligonucleotide probe comprises one or more deoxyribonucleic acid (DNA) residues.

9. The method of claim 2 or 7, wherein the oligonucleotide probe comprises a central segment which is comprised of DNA residues flanked by 5′ and 3′ terminal segments which each comprise LNA residues.

10. The method of claim 9, wherein the central segment is comprised of between 5 and 20 DNA residues.

11. The method of claim 10 or 11, wherein the 5′ and 3′ terminal segments are independently comprised of between 2 and 8 LNA residues.

12. The method of claim 2, wherein step (c) comprises incubating the substrate with only one single-strand-specific nuclease.

13. The method of claim 2, wherein step (c) comprises incubating the substrate with two or more different single-strand-specific nucleases.

14. The method of claim 13, wherein the substrate is incubated with the two or more different single-strand-specific nucleases sequentially and the substrate is washed after each incubation.

15. The method of claim 13, wherein the substrate is incubated with the two or more single-strand-specific nucleases simultaneously.

16. The method of claim 2, wherein the one or more single-strand-specific nucleases comprises micrococcal nuclease.

17. The method of claim 2, wherein the one or more single-strand-specific nucleases comprises mung bean nuclease.

18. The method of claim 2, wherein the one or more single-strand-specific nucleases comprises micrococcal nuclease and mung bean nuclease.

19. The method of claim 11, wherein the only one single-strand-specific nuclease is mung bean nuclease.

20. The method of claim 13 or 14, wherein the two or more different nucleases comprise micrococcal nuclease and mung bean nuclease.

21. The method of claim 1 or 2, wherein the sample is obtained from a tissue and the method further comprises a step of quantifying a level of PMO in the tissue based on a level of the detectable signal detected in step (e).

22. The method of claim 21, wherein the tissue is selected from blood, kidney, liver, gastrointestinal, lung, muscle, spleen, brain, spinal cord, or a combination thereof.

23. The method of claim 22, wherein the blood tissue is or comprises plasma or serum.

24. The method of claim 22, wherein the muscle tissue is diaphragm, gastrocnemius, biceps, tibialis anterior (TA), heart, and/or quadriceps.

25. The method of claim 1 or 2, wherein the sample comprises a PMO which comprises a sequence of 20 to 30 contiguous nucleotides and has a nucleotide sequence selected from at least one exon of a mammalian dystrophin gene.

26. The method of claim 25, wherein at least one exon is selected from exon 23, 44, 45, 46, 51, or 53.

27. The method of claim 25 or 26, wherein the mammalian dystrophin gene is a human dystrophin gene.

28. The method of any one of claims 2-27, wherein the PMO has previously been delivered to a patient, and wherein the PMO was conjugated to a peptide (P-PMO) for delivery.

29. A method of assessing the tissue distribution of a P-PMO, the method comprising performing the method of any one of the preceding claims on one or more samples that have been obtained from one or more tissues of a subject to whom the P-PMO has been administered.

30. The method of claim 29, wherein the method is performed on two or more samples that have been obtained from two or more tissues of the subject.

31. The method of claim 29 or 30, wherein the method is repeated for a different P-PMO.

32. A method of assessing the ability of a P-PMO to modulate expression of a target protein, the method comprising performing the method of any one of the preceding claims on a sample that has been obtained from a tissue of a subject to whom a P-PMO has been administered and comparing the level of a PMO derived from the P-PMO in the tissue with a level of the target protein in the tissue.

33. The method of claim 32, wherein the method is performed on two or more samples that have been obtained from two or more tissues of the subject.

34. The method of claim 32 or 33, wherein the method is repeated for a different P-PMO.

35. The method of any one of claims 32-34, wherein the target protein is dystrophin.


SEQUENCE LISTING (3 sequences)

SEQ ID NO: 1 - DNA, 25 bp, Artificial Sequence
  Description of Artificial Sequence Synthetic oligonucleotide
ggccaaacctcggcttacctgaaat

SEQ ID NO: 2 - DNA, 19 bp, Artificial Sequence
  Description of Artificial Sequence Synthetic probe
ttcaggtaagccgaggttt

SEQ ID NO: 3 - DNA, 25 bp, Artificial Sequence
  Description of Artificial Sequence Synthetic probe
atttcaggtaagccgaggtttggcc